(19) United States
(12) Patent Application Publication
(10) Pub. No.: US 2023/0321135 A1
(43) Pub. Date: Oct. 12, 2023

(54) TARGETING OF LACTATE DEHYDROGENASE C, METHODS OF PREPARING SAME, AND METHODS OF USING SAME IN COMBINATION WITH ANTI-CANCER TREATMENTS

(71) Applicant: QATAR FOUNDATION FOR EDUCATION, SCIENCE AND COMMUNITY DEVELOPMENT, Dohar (QA)

(72) Inventors: Julie Valerie Caroline M. Decock, Doha (QA); Adviti Naik, Doha (QA)

(21) Appl. No.: 18/009,245

(22) PCT Filed: Jun. 8, 2021
(86) PCT No.: PCT/QA2021/050012

§ 371 Date: Dec. 8, 2022

Related U.S. Application Data
(60) Provisional application No. 63/036,097, filed on Jun. 8, 2020.

(51) Int. Cl. A61K 31/713; A61K 33/243; C12N 15/113; A61P 35/00

(52) U.S. Cl. CPC A61K 31/713; A61K 33/243; C12N 15/1137; A61P 35/00; C12N 2310/14

(57) ABSTRACT

The present disclosure provides a therapeutic method comprised of targeting the tumor-associated antigen, Lactate Dehydrogenase C (LDHC), in LDHC-expressing cancers or tumors. This novel approach offers a highly specific cancer treatment with little or no off-target effects. Targeting LDHC with the use of silencing molecules increases tumor cell death by inducing excess DNA damage, and more importantly, can render breast tumor cells more responsive to non-specific, DNA damaging drugs and DNA damage repair inhibitors, such as platinum-based alkylating agents (e.g. cisplatin) and Poly ADP-Ribose Polymerase (PARP) inhibitors (e.g. olaparib). Targeting LDHC in tumor cells has the potential to specifically sensitize tumor cells to these drugs and/or inhibitors, leading to enhanced tumor cell killing and potentially minimizing side effects.

### CROSS REFERENCE TO RELATED APPLICATION

This application claims the benefit of U.S. provisional patent application No. 63/036,097, filed Jun. 8, 2020, the entire disclosure of which is incorporated herein by reference.

### TECHNICAL FIELD

The present disclosure generally relates to the method of targeting Lactate Dehydrogenase C (LDHC) for use in cancer therapies. The present disclosure relates to methods of using various targeted silencing and/or inhibiting techniques against LDHC-expressing cancer cells. The present disclosure also relates to methods of preparing and administering LDHC targeting molecules in vivo using various delivery systems. The present disclosure also relates to the use of LDHC targeting methods alone or in combination with other therapies or treatments for LDHC-expressing cancers and other related diseases and disorders.

### BACKGROUND

Tumor cells maintain a fine balance between genomic instability, to drive genetic variations, and genomic integrity to allow the survival and propagation of malignant cells. Cancer Testis Antigens (CTAs), a class of germ cell-specific proteins with re-expression in tumors, play a critical role in maintaining genomic integrity during meiosis in spermatocytes. This characteristic is exploited by CTA-expressing tumor cells to promote survival. Hence, targeting CTAs could exacerbate genomic instability and potentially lead to tumor cell death with minimal off-target effects. Lactate dehydrogenase C (LDHC; UniProtKB: P07864; OMIM: 150150) is an immunogenic CTA with tumor-specific expression, and is a member of the lactate dehydrogenase family of enzymes involved in metabolic reprogramming. These enzymes catalyze the interconversion of pyruvate and L-lactate as well as play important roles in aerobic glycolysis. The LDHC gene encoding LDHC is located on chromosome 11 and consists of about 38,941 base pairs (HGNC: 6544; Entrez Gene: 3948; Ensembl: ENSG00000166796; NG_011816).

Although the oncogenic role of LDHC in tumor cell motility has been reported, its function in sustaining genomic stability is not known. LDHC expression is restricted to mature testis and spermatozoa. Although LDHC expression is suppressed in normal somatic tissues, it has been found to be expressed in various malignant tissues, making its expression highly tumor-specific. Specifically, LDHC expression has been detected in different tumor types at varying degrees with frequencies such as up to 100% in lung adenocarcinoma, 83% in cervical cancer, 76% in high-grade serous ovarian carcinoma (HGSC), 44% in melanoma, and 35% in breast cancer.

The tumor-specific expression of LDHC is thought to be related to the metabolic reprograming of cancer cells and/or cancer cell migration and invasion. The production of lactate by LDHC and the concomitant tumor acidity likely negatively influence the anti-tumor immune response by skewing the immune cell compartment towards an immunosuppressive environment.

Breast cancer remains the most common cancer among women, accounting for 30% of all cancer cases and is the leading cause of female cancer-related deaths. Triple negative breast cancer (TNBC), representing 15-20% of breast tumors, is associated with poor clinical outcome, early relapse and excess mortality accounting for almost one third of breast-cancer related deaths. The inventors have identified LDHC as a promising novel target for cancer therapy.

### SUMMARY

Disclosed herein are methods to sensitize cancer cells to anti-cancer treatments by targeting LDHC in LDHC-expressing tumors or cancer cells.

Described herein are methods of treatment for use in patients with LHDC-expressing cancer cells or tumors; the treatment method comprises administering LDHC targeting molecules that can specifically target LDHC-expressing cells. The LDHC targeting molecules can be used alone or can be administered in combination with other cancer therapies or cancer treatments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E depict LDHC expression in breast cancer. FIG. 1A depicts a box plot of LDHC mRNA expression in tumor and normal breast tissue using the Breast Cancer (BRCA) TCGA dataset. Differential expression analyzed with one-way ANOVA. FIG. 1B depicts a box plot depicting LDHC mRNA expression in intrinsic breast cancer subtypes in TCGA dataset. Statistical analysis performed using ANOVA and Tukey post hoc test, ***basal-like vs luminal A. FIG. 1C depicts Kaplan-Meier curves for overall and disease-specific survival, stratified by median LDHC mRNA expression in the METABRIC cohort. The log rank test was used to assess survival differences. Hazards ratio (HR) and p values are indicated. Figure D: LDHC mRNA, normalized to RPLPO, and LDHC protein expression in breast cancer cell lines stably transfected with shCTRL or shLDHC expression vectors. β-actin protein expression indicated as western blot loading control. Numbers under each lane represent mean densitometry values (arbitrary units) of LDHC signal normalized to β-actin from three independent experiments. FIG. 1E: (Top) Flow cytometry-based quantification of giant cells. Left-representative forward scatter (FSC-A) vs side scatter (SSC-A) flow cytometry plots for MDA-MB-468 cells with cell populations sub-grouped as P1 and P2 (giant cells). Right-frequency of giant cells (P2) in LDHC-silenced compared to control cells. (Bottom) representative immunofluorescence microscopy images of MDA-MB-468 with nuclear (DAPI, blue) and F-actin (red) staining (20× magnification), inserts at 2× zoom. Statistical analysis comparing shCTRL vs shLDHC performed using Student's t-test. Error bars represent standard error of mean (±SEM) from three independent replicates. *p≤0.05, **p≤0.01, ***p≤0.001.

FIGS. 2A-2C depict how silencing LDHC exacerbates polyploidy and nuclear aberrations. FIG. 2A depicts Frequency of cells exhibiting polyploidy (≥4N ploidy) in shLDHC versus shCTRL cells, as determined by PI flow cytometry analysis. FIG. 2B depicts the degree of polyploidy in shLDHC versus shCTRL MDA-MB-468 cells. Representative histograms with inserts representing genomic content of giant cells (P2). FIG. 2C: (Top) Frequency of nuclear aberrations in shLDHC and shCTRL cells (n=600). (Bottom) Representative immunofluorescence microscopy images of nuclear aberrations in MDA-MB-468 shCTRL and shLDHC cells with nuclear (DAPI, blue) and β-tubulin (red) staining (100× magnification), inserts at 2.5× zoom. All statistical analysis comparing shCTRL vs shLDHC performed using Student's t-test. Error bars represent standard error of mean (±SEM) from three independent replicates. *p≤1.05, **p≤1.01, ***p≤1.001. MC-Mitotic catastrophe, MN-Micronuclei, MNC-Multinucleated cells, NBUD-Nuclear budding, NPB-Nucleoplasmic bridges.

FIGS. 3A-3E depict how LDHC regulates DNA damage accumulation and microtubule network stability. FIG. 3A: (Top) Representative immunofluorescence microscopy images of phospho-γ-H2AX (red) and DAPI (blue)-stained nuclei in MDA-MB-468 cells (100x magnification). (Bottom) Western blot of phospho-γ-H2AX and β-actin as loading control. FIG. 3B depicts a western blot of full length and degraded α-tubulin expression. Note that HCC-1500 cells show an additional degraded product of α-tubulin with slightly higher molecular weight (not quantified). FIG. 3C: Representative immunofluorescence microscopy images of acetylated α-tubulin (red) and cell nuclei (DAPI, blue) in MDA-MB-468 cells (100× magnification). FIG. 3D: MAP1B mRNA expression, normalized to RPLPO expression. FIG. 3E: Representative immunofluorescence microscopy images of α-tubulin (green), MAP1B (red) and DAPI (blue)-stained nuclei in MDA-MB-468 cells (100× magnification). For panels A and B western blots, numbers under each lane represent mean densitometry values (arbitrary units) for respective protein signal normalized to β-actin from three independent experiments. Statistical analysis comparing shCTRL vs shLDHC performed using Student's t-test. Error bars represent standard error of mean (±SEM) from three independent replicates. ***p≤0.001.

FIGS. 4A-4E depict how LDHC abrogation induces apoptosis, mitotic dysregulation and decreases long-term survival. FIG. 4A: (Top) Western blot of cleaved caspase 3 expression with β-actin as loading control. Numbers under each lane represent mean densitometry values (arbitrary units) for cleaved caspase 3 signal normalized to β-actin from three independent experiments. (Bottom) Quantification of apoptotic cells by AnnexinV/PI flow cytometry with early apoptosis defined as AnnV+PI− cells and late apoptosis as AnnV+PI+ cells. FIG. 4B depicts time-course of cell cycle distribution of synchronized MDA-MB-468 cells using PI flow cytometry (error bars represent±standard deviation). FIG. 4C: (Top) Representative cell cycle profile of asynchronous cell populations, as determined by EdU/PI flow cytometry. (Bottom) Proportion of asynchronous MDA-MB-468 and BT-549 cells in each cell cycle phase. FIG. 4D depicts a line chart representing the fold change (FC) in protein expression over time, as determined by western blot of cyclin B1, phopho-cdc2, phosph-γ-H2AX, cleaved caspase 3 expression with representative β-actin as loading control. Mean densitometry values (arbitrary units) of respective protein signals from three independent experiments were normalized to β-actin, and the normalized values were used to calculate the fold change in comparison to shCTRL at Oh. FIG. 4E: (Top left) Mean OD590 for crystal violet quantification, (Bottom left) mean number of colonies, and (Right) representative image as determined by clonogenic assay. All statistical analysis comparing shCTRL vs shLDHC performed using Student's t-test. Error bars (in A, C, E) represent standard error of mean (±SEM) from three independent replicates. *p≤1.05, **p≤1.01, ***p≤1.001.

FIGS. 5A-5C depict LDHC and cell cycle regulation of MDA-MB-468 cells. FIG. 5A depicts GO enrichment of differentially expressed genes after LDHC silencing as determined by the Cell Cycle RT2 Profiler qPCR array. FIG. 5B depicts a western blot of cell cycle regulator protein expression with β-actin as loading control. Numbers under each lane represent mean densitometry values (arbitrary units) for respective protein signal normalized to β-actin from three independent experiments. FIG. 5C depicts a representative immunofluorescence microscopy images of BubR1 (red) and DAPI-stained nuclei (blue) in prometaphase, metaphase and anaphase stages of mitosis (indicated by white arrows, 100× magnification). All statistical analysis comparing shCTRL vs shLDHC performed using Student's t-test. Error bars represent standard error of mean (±SEM) from three independent replicates with n=90 cells for each condition. ***p≤1.001.

FIGS. 6A-6D depict how LDHC silencing improves sensitivity of MDA-MB-468 cells to DNA damage inducers and DNA damage repair inhibitors. FIG. 6A: Annexin V/PI flow cytometric quantification of apoptosis after 72 hrs of treatment with olaparib or cisplatin. FIG. 6B depicts a western blot of cleaved caspase 3 protein expression with β-actin as loading control. Numbers under each lane represent mean densitometry values (arbitrary units) for cleaved caspase 3 signal (high exposure blot) from three independent experiments normalized to β-actin. FIG. 6C: Representative immunofluorescence microscopy images of phospho-γ-H2AX (red) and DAPI-stained nuclei (100× magnification). Yellow arrows indicate early apoptotic cells and white arrows indicate late apoptotic cells. FIG. 6D: (Left) Mean OD590 for crystal violet quantification, (Middle) mean number of colonies and (Right) representative images taken at 14 days of culture post-treatment (72 h). All statistical analysis comparing shCTRL vs shLDHC performed using Student's t-test. Error bars represent standard error of mean (±SEM) from three independent replicates. *p≤1.05, **p≤1.01.

FIG. 7 depicts a schematic model of the effect of LDHC targeting on cell cycle checkpoints and cell fate. Silencing of LDHC in breast cancer cells increases the rate of mitotic entry by dysregulation of several cell cycle checkpoints, resulting in a shorter cell cycle. Firstly, targeting LDHC shortens the G1 phase and downregulates several molecules controlling the G1/S checkpoint and mediates override of the intra S-phase checkpoint. Next, G2/M checkpoint regulators are aberrantly expressed in LDHC silenced cells. Together, this results in excessive DNA damage (black lines) due to the decreased expression of DNA damage sensors upstream of the DDR pathway and hence, lack of functional repair mechanisms. On mitotic entry, the unstable microtubule network in LDHC silenced cells triggers defective chromosome segregation, thus activating the spindle assembly checkpoint (SAC). While a proportion of cells with high levels of genomic instability undergo arrest and cell death, an additional population of cells with lower levels of instability undergo long-term SAC activation and mitotic slippage in the absence of cytokinesis. These cells form giant polyploid cancer cells that subsequently undergo cell death or senescence, ultimately diminishing clonogenicity or long-term survival of cancer cells. Additionally, targeting LDHC in combination with DNA-damaging/DNA-repair inhibiting agents (orange arrows) synergistically dysregulates the DNA damage repair pathways and promotes cell death pathways, resulting in significant loss of cell survival. The G1/S, intra-S, G2/M and SAC checkpoints are depicted as red lines. The molecular regulators depicted were found to be involved in LDHC silenced MDA-MB-468, however, we cannot exclude that there may be cell-line dependent differences in regulators.

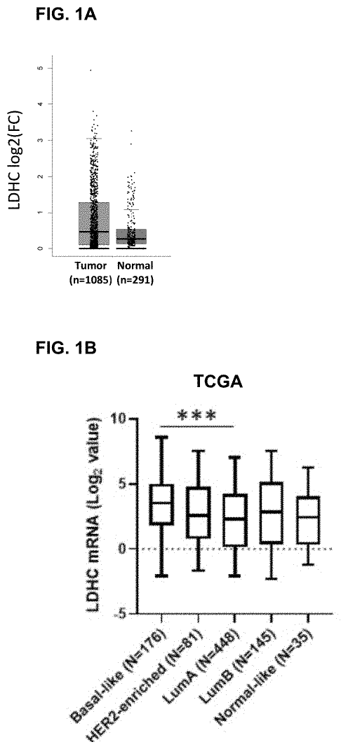

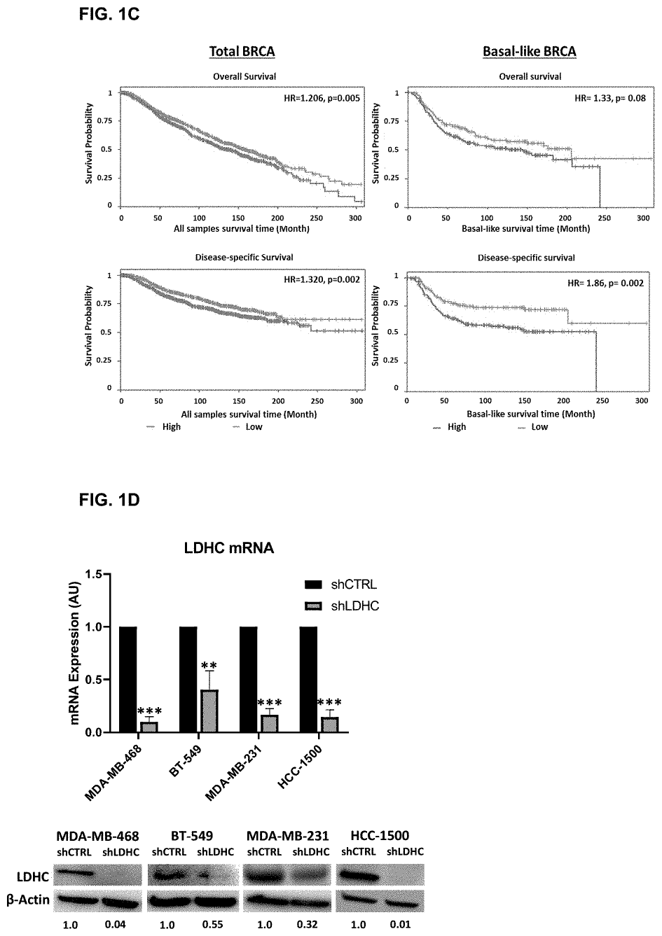

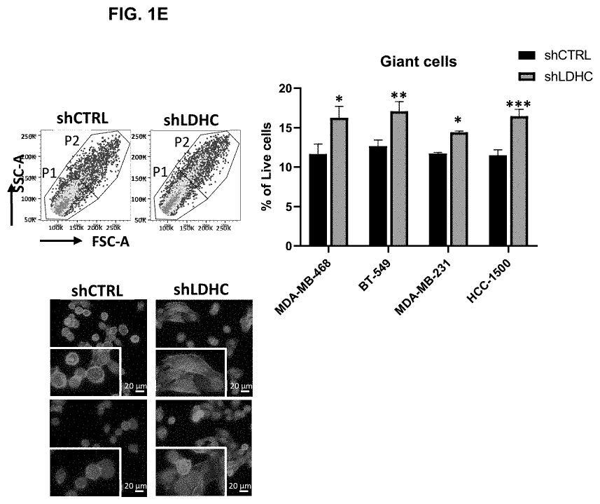

Supplemental FIGS. 1A-1C depict LDHC expression across normal tissues. Supplemental FIG. 1A depicts normalized LDHC mRNA expression in 55 tissue types and 6 blood cell types, using a consensus dataset consisting of three transcriptomic datasets (HPA, GTEx and FANTOM5) and internal normalization pipeline. Color-coding represents tissue groups with common functional features. Supplemental FIG. 1B depicts LDHC protein expression in 44 normal tissues as reported by the Human Protein Atlas. Supplemental FIG. 1C illustrates that the α-tubulin subunit of microtubules in shLDHC breast cancer cells revealed an increase in expression of its truncated form. Supplemental FIG. 1D depicts LDHC mRNA expression determined by qRT-PCR and normalized by RPLPO.

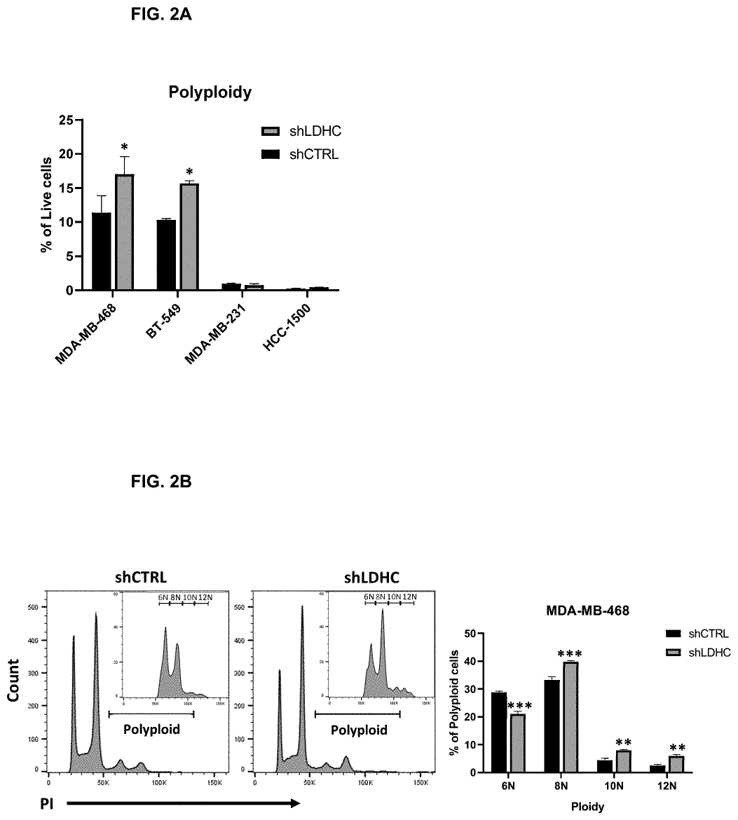

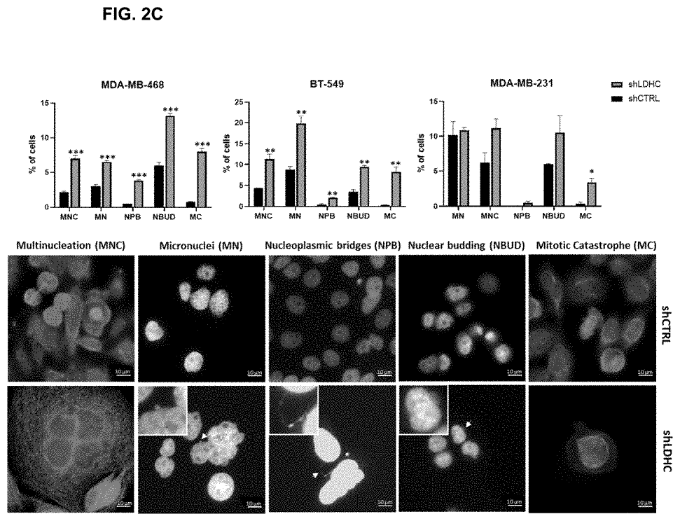

Supplemental FIGS. 2A-2E depict how LDHC silencing does not alter LDHA/LDHB expression, and increases DNA damage and apoptosis. Supplemental FIG. 2A depicts a western blot of LDHA and LDHB, with β-actin as loading control. Supplemental FIG. 2B depicts a western blot of DNA damage sensors. Numbers under each lane represent mean densitometry values (arbitrary units) normalized to β-actin from three independent experiments. Supplemental FIG. 2C depicts a western blot of cleaved caspase 3 in M DA-M B-231 and HCC-1500 shCTRL and shLDHC cells. Numbers under each lane represent mean densitometry values (arbitrary units) normalized to β-actin from three independent experiments. Supplemental FIG. 2D depicts a representative AnnexinV/PI flow cytometry plots of MDA-MB-468 shCTRL and shLDHC cells. Supplemental FIG. 2E depicts Annexin V/PI flow cytometric quantification of MDA-MB-231 and HCC-1500 shCTRL and shLDHC cells. Statistical analysis comparing shCTRL vs shLDHC performed using Student's t-test. Error bars represent standard error of mean (±SEM) from three independent replicates. *p≤0.05, **p≤0.01.

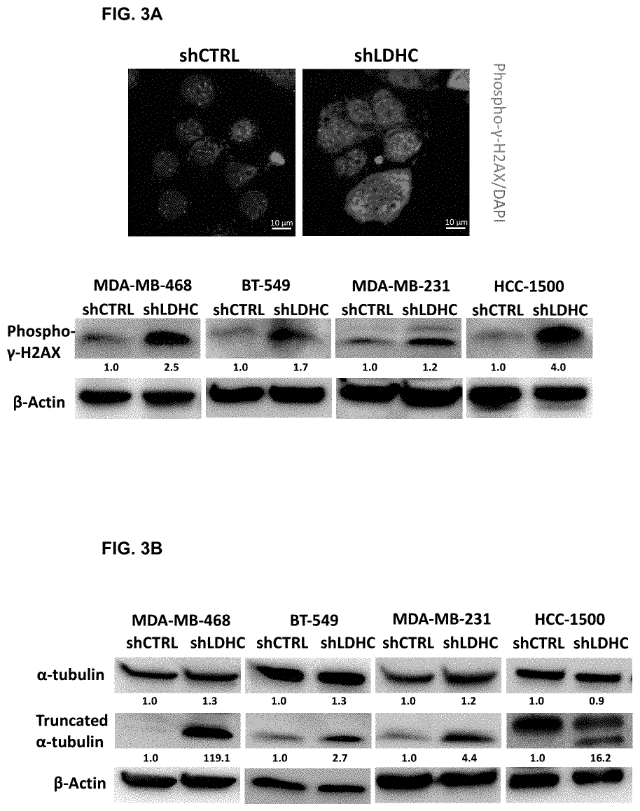

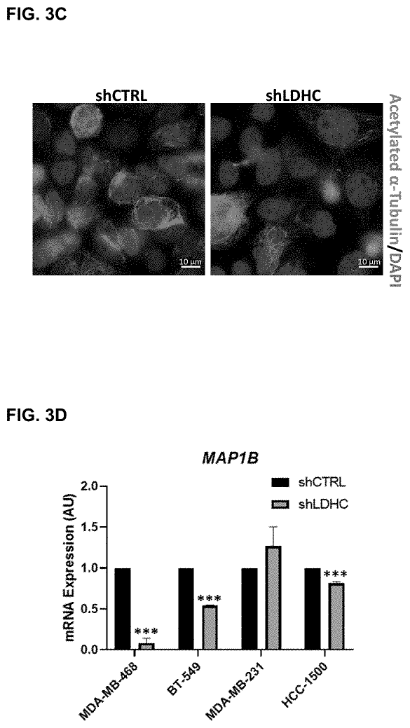

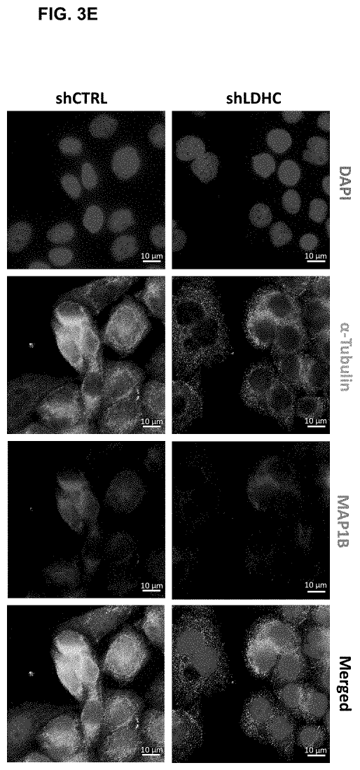

Supplemental FIGS. 3A-3C depict aberrant cell cycle upon LDHC silencing. Supplemental FIG. 3A depicts a representative time-course of cell cycle histograms in synchronized MDA-MB-468 and BT-549 cells using PI flow cytometry. Supplemental FIG. 3B: (Left) Time-course of cell cycle distribution of synchronized BT-549 cells using PI flow cytometry (error bars represent±standard deviation). (Right) Frequency of BT-549 cells with polyploidy over a time-course of 12 h. Supplemental FIG. 3C depicts Quantification of sub-G1 cell population at 12 h post thymidine block release. Statistical analysis comparing shCTRL vs shLDHC performed using Student's t-test. Error bars represent standard error of mean (±SEM) from three independent replicates. *p≤1.05, ***p≤1.001.

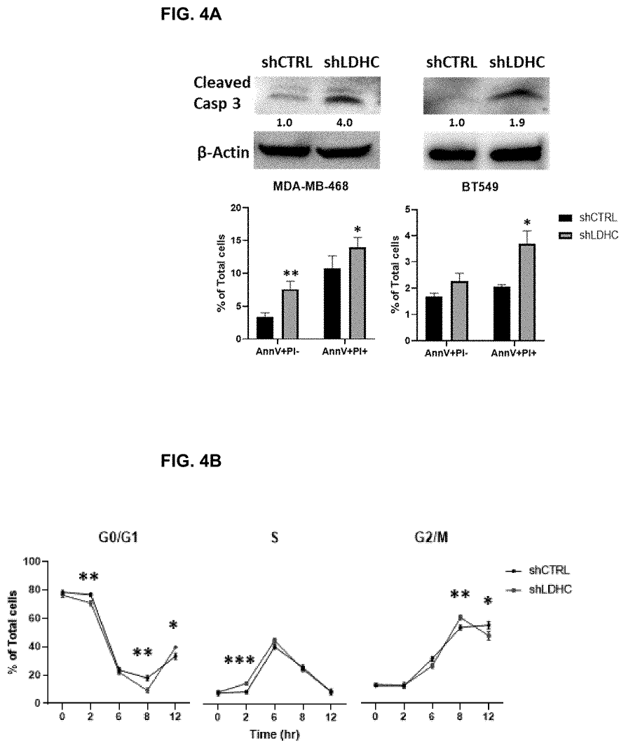

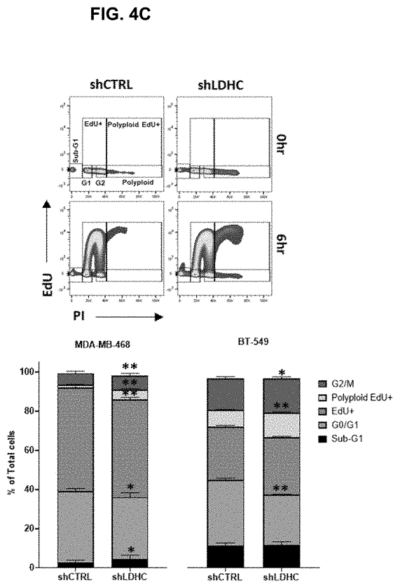

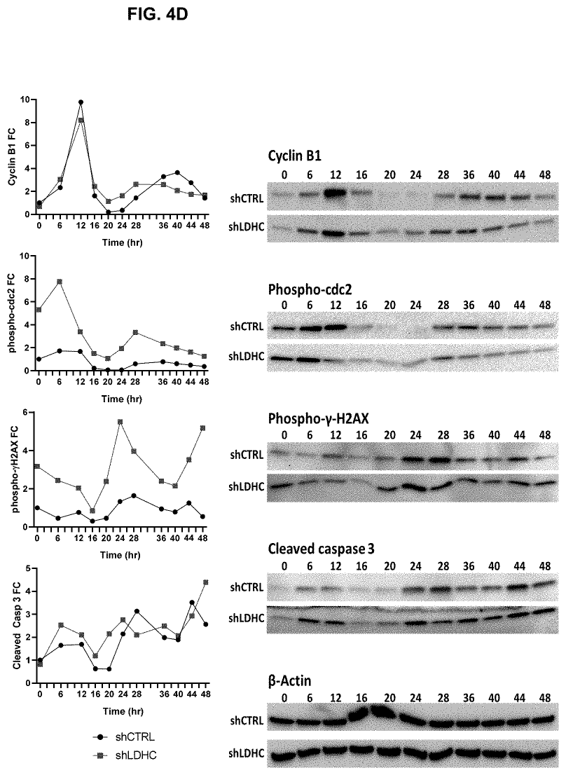

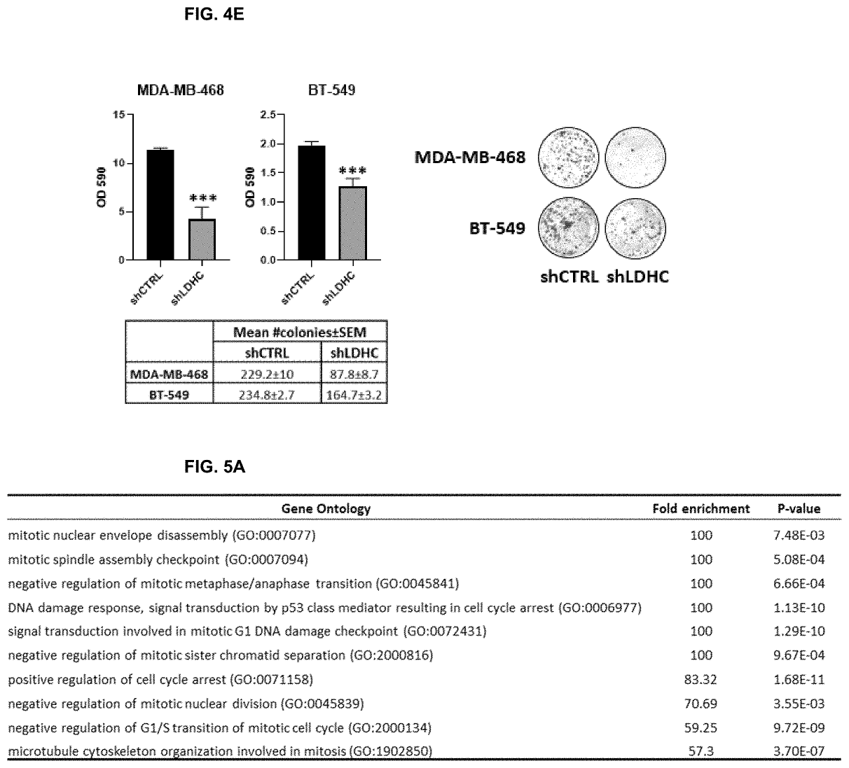

Supplemental FIGS. 4A-4C depict aberrant cell cycle and cell fate upon LDHC silencing. Supplemental FIG. 4A depicts a representative asynchronous cell cycle profile of MDA-MB-231 and HCC-1500 cells, determined by EdU/PI flow cytometry. Supplemental FIG. 4B: visualization of senescence, determined by β-galactosidase staining. Supplemental FIG. 4C depicts crystal violet quantification at OD590 and representative image of MDA-MB-231 and HCC-1500 clonogenic assay. Statistical analysis comparing shCTRL vs shLDHC performed using Student's t-test. Error bars represent standard error of mean (±SEM) from three independent replicates. *p≤0.05, **p≤0.01, ***p≤0.001.

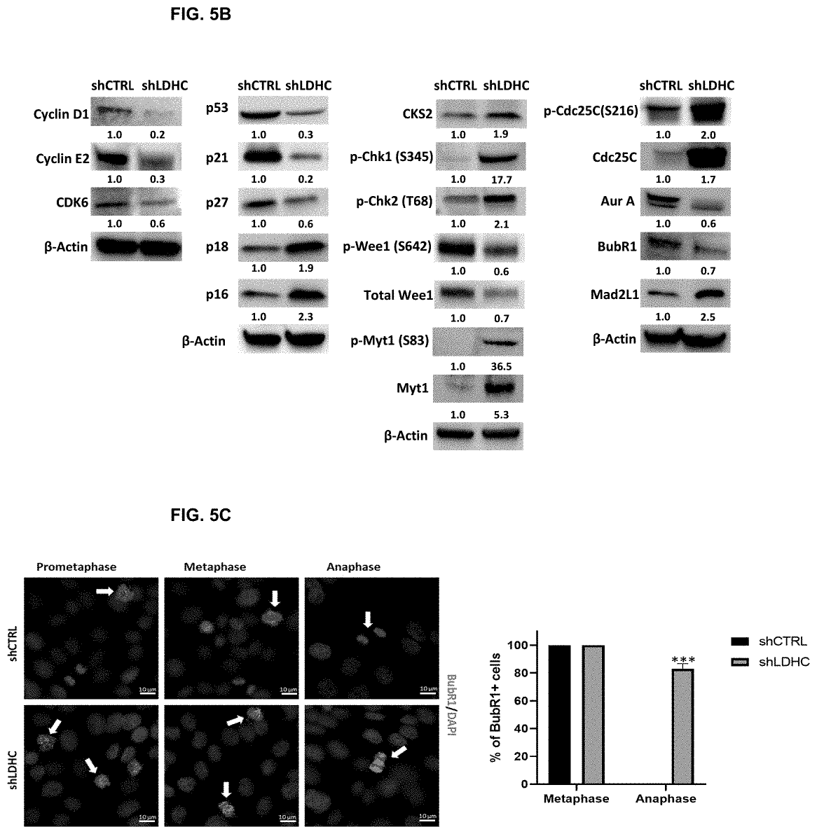

Supplemental FIGS. 5A-5D depict how LDHC silencing improves sensitivity of breast cancer cells to DNA damage inducers and DNA damage repair inhibitors. Supplemental FIG. 5A depicts a representative Annexin V/PI flow cytometry plots of MDA-MB-468 cells after 72 hrs of treatment with olaparib or cisplatin. Supplemental FIG. 5B depicts Annexin V/PI flow cytometric quantification of BT-549 cells after 72 hrs of treatment with olaparib or cisplatin. Supplemental FIG. 5C: Annexin V/PI flow cytometric quantification of HCC-1500 cells after 72 hrs of treatment with olaparib or cisplatin. Supplemental FIG. 5D: BT-549, HCC-1500 and MDA-MB-231 clonogenic assay at 14 days of culture post-treatment (72 h). Cisplatin treatment of HCC-1500 completely abolished clonogenic ability of shCTRL and shLDHC cells and hence is not depicted in the figure. Statistical analysis comparing shCTRL vs shLDHC performed using Student's t-test. Error bars represent standard error of mean (±SEM) from three independent replicates. *p≤1.05, **p≤1.01, ***p≤1.001.

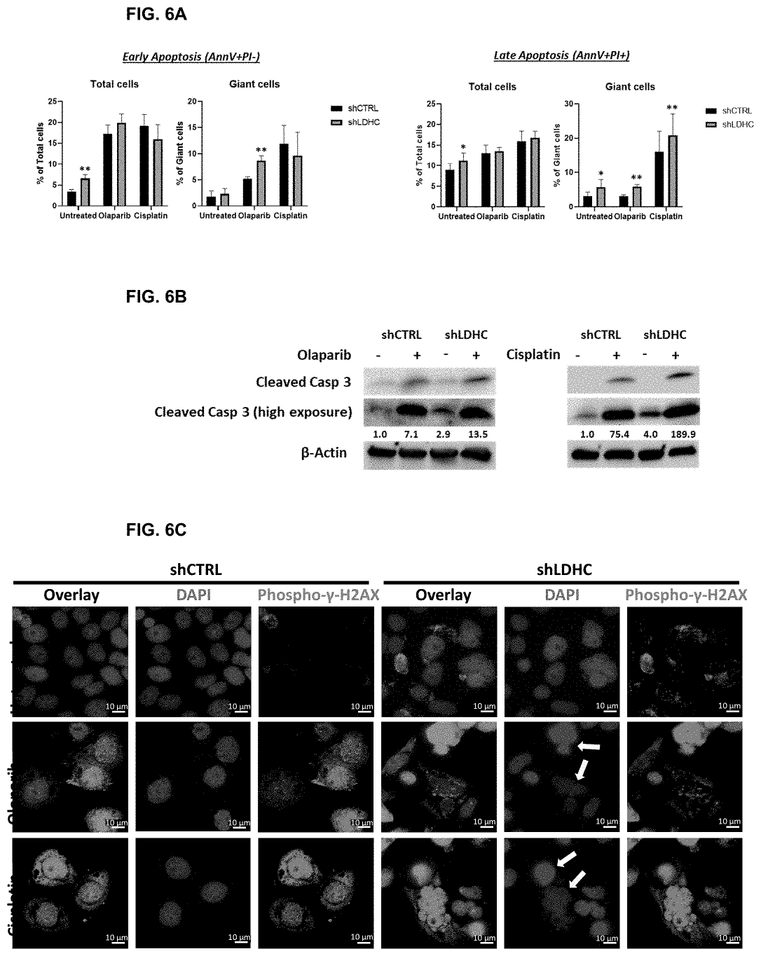

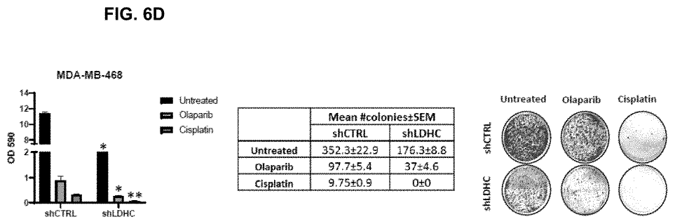

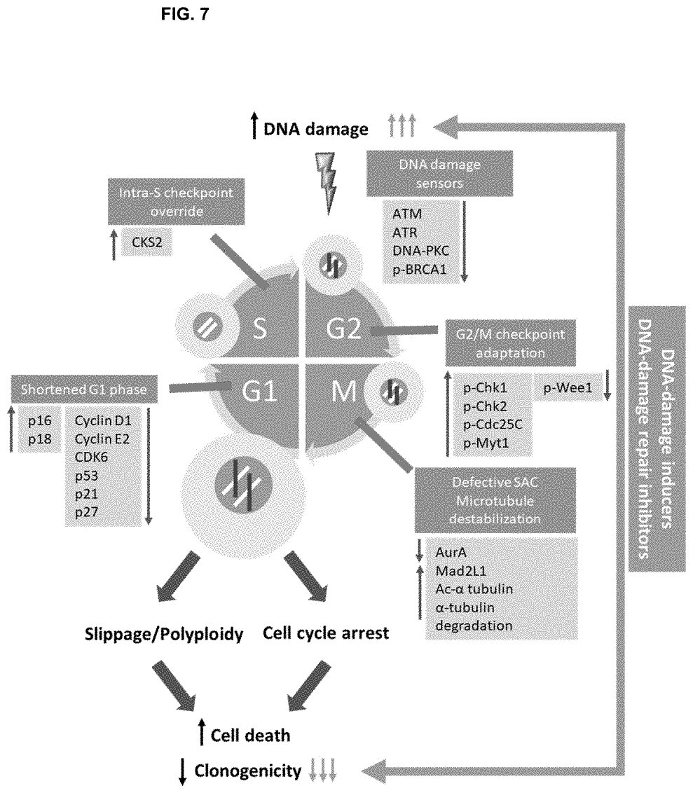

Supplemental FIG. 6 depicts LDHC expression in TP53 mutant breast cancer. Box plot of LDHC mRNA expression in normal breast and TP53 mutant and non-mutant (wild-type) tumors using the TOGA Breast Cancer dataset. Data was retrieved and visualized using the UALCAN web-portal (available at http://ualcan.path.uab.edu). **p≤0.01, ***p≤0.001.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including,” “containing” and “having” should all be construed to be inclusive, unless such a construction is clearly prohibited from the context. Further in this regard, these terms specify the presence of the stated features but not preclude the presence of additional or further features.

Nevertheless, the compositions and methods disclosed herein may lack any element that is not specifically disclosed herein. Thus, a disclosure of an embodiment using the term “comprising” is (i) a disclosure of embodiments having the identified components or steps and also additional components or steps, (ii) a disclosure of embodiments “consisting essentially of” the identified components or steps, and (iii) a disclosure of embodiments “consisting of” the identified components or steps. Any embodiment disclosed herein can be combined with any other embodiment disclosed herein.

The term “and/or” used in the context of “X and/or Y” should be interpreted as “X,” or “Y,” or “X and Y.” Similarly, “at least one of X or Y” should be interpreted as “X,” or “Y,” or “X and Y.”

Where used herein, the terms “example” and “such as,” particularly when followed by a listing of terms, are merely exemplary and illustrative and should not be deemed to be exclusive or comprehensive.

The term “isolated” means altered or removed from a natural state. For example, a protein naturally present in a living animal is not “isolated,” while the same protein partially or completely separated from all or some of the coexisting materials of its natural state is “isolated”.

A “subject” or “individual” is a mammal, preferably a human. As used herein, an “effective amount” is an amount of a compound, formulation, material, composition, or a combination of such, that treats a disease or medical condition in an individual, or, more generally, reduces symptoms, manages progression of the disease or disorder or reduces the disease burden. Herein, “effective amount” and “therapeutically effective amount” are used interchangeably.

The terms “peptide” or “protein” or “polypeptide” refers to a polymer of amino acid residues covalently linked by peptide bonds. The terms “peptides” or “proteins” or “polypeptides,” used herein, may also refer to a polymer of amino acids where one or more of the amino acids may be a modified residue, such as an artificial amino acid mimetic or a synthetic amino acid residue. The terms “peptide” or “protein” or “polypeptide” are used interchangeably.

The term “segment,” when used in reference to a “peptide” or “protein” or “polypeptide,” refers to the entire sequence and in addition, optionally, refers to a portion of that “peptide” or “protein” or “polypeptide” that is at least one or more of the amino acids, but less than the entire sequence of the “peptide” or “protein” or “polypeptide.”

The term “antigen” or “immunogen” or “hapten” is a substance or structure or molecule, such as a “peptide” or “protein” or “polypeptide,” that is or is perceived to be foreign to the body and evokes an immune response alone or after forming a complex with a larger molecule. A “protein” or “peptide” that evokes or provokes an immune response can be used interchangeably with the term “antigen.” One of ordinary skill in the art will understand that any “protein” or “peptide” or “polypeptide” that elicits an immune response, and is therefore also an “antigen,” may not require the full length “protein” or “peptide” or “polypeptide” sequence to elicit an immune response. Therefore, a “portion” or “segment” of the “protein” or “peptide” or “polypeptide” sequence may also be suffice as an “antigen.”

The term “amino acid” refers to both naturally occurring and synthetic amino acids as well as analogs and amino acid mimetics. Herein, amino acids are referred to by the standard IUB/IUPAC amino acid codes, including both one-letter and three-letter codes.

The term “vector” refers to a “DNA vector” or “RNA vector” or a “plasmid” or a “lentivirus vector” or an “adenoviral vector” or a “retrovirus vector” or any other type of vector known to one of ordinary skill in the art.

The terms “nucleic acid” or “genetic material” or “polynucleotide” refers to “deoxyribonucleic acid” (DNA) or “ribonucleic acid” (RNA) and polymers thereof, in either single- or double-stranded form. The DNA or RNA or polymers thereof, refers to both naturally occurring and synthetic forms.

The terms “siRNA” and “shRNA” refer to short RNA molecules with a specific complementary sequence to some or all isoforms of a specific mRNA.

The terms “nanoparticles”, “nanoplexes” and “nanostructures” refer to nanoscale delivery systems and complexes thereof with natural or synthetic polymers.

The terms “liposomes” and “lipoplexes” refer to delivery systems composed of lipids and complexes thereof with natural or synthetic polymers.

The term “DNA scaffolds” refers to delivery systems composed of deoxyribonucleic acid nanostructures.

The term “tumor-homing peptide” refers to short amino acid sequences that can specifically recognize tumor cells or tumor-specific components.

The term “biologically-responsive” refers to properties of the delivery system that enable release of the encapsulated load on recognition of tumor microenvironment-specific stimuli.

The term “exosomes” refers to delivery systems comprising cell-derived extracellular vesicles or their synthetic derivatives.

The term “nanobody” refers to a single-domain antibody fragment that binds to a specific antigen, composed of naturally-occurring or synthetic heavy chain only antibodies and modifications thereof.

The term “antibody” refers to a protein comprising of four polypeptides forming two identical heavy chains and two identical light chains that can recognize a specific antigen.

The term “inhibitor” refers to a chemically synthesized or biologically derived macromolecule or compound capable of blocking the activity or function of a protein.

The terms “treatment” and “treat” include both prophylactic or preventive treatment (that prevent and/or slow the development of a targeted pathologic condition, infection, disorder, or disease) and curative, therapeutic or disease-modifying treatment, including therapeutic measures that cure, slow down, lessen symptoms of, and/or halt progression of a diagnosed pathologic condition, infection, disorder, or disease. The terms “treatment” and “treat” do not necessarily imply that a subject is treated until total recovery. The terms “treatment” and “treat” are also intended to include the potentiation or otherwise enhancement of one or more primary prophylactic or therapeutic measures. As non-limiting examples, a treatment can be performed by a doctor, a healthcare professional, a veterinarian, a veterinarian professional, or another human.

The term “unit dosage form,” as used herein, refers to physically discrete units suitable as unitary dosages for subjects, each unit containing a predetermined quantity of the composition disclosed herein in amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

The terms “tumor” or “tumour” or “cancer” are used interchangeably. “Cancer” is a disease characterized by uncontrolled growth of cells. The embodiments disclosed herein may target any type of cancer that expresses LDHC or a similar molecule.

The term “sterile” is understood to mean free from any bacteria or other living microorganisms.

The term “pharmaceutically acceptable” as used herein refers to substances that do not cause substantial adverse allergic or immunological reactions when administered to a subject.

All percentages expressed herein are by weight of the total weight of the composition unless expressed otherwise. When reference herein is made to the pH, values correspond to pH measured at about 25° C. with standard equipment. “Ambient temperature” or “room temperature” is between about 15° C. and about 25° C., and ambient pressure is about 100 kPa.

The term “mM”, as used herein, refers to a molar concentration unit of an aqueous solution, which is mmol/L. For example, 1.0 mM equals 1.0 mmol/L.

The terms “substantially no,” “essentially free” or “substantially free” as used in reference to a particular component means that any of the component present constitutes no more than about 3.0% by weight, such as no more than about 2.0% by weight, no more than about 1.0% by weight, preferably no more than about 0.5% by weight or, more preferably, no more than about 0.1% by weight.

### Embodiments

The present disclosure generally relates to a method of treating patients with LDHC-expressing cancer. The method comprising targeted silencing of LDHC-expressing cancer cells or tumors.

Various non-exhaustive, non-limiting aspects of methods for treating patients with LDHC-expressing cancers according to the present disclosure may be used alone or in combination with one or more other aspects described herein. Without limiting the foregoing description, in some embodiments of the present disclosure, a method comprising administering a composition to a subject with LDHC-expressing cancer, wherein the composition comprises LDHC targeting molecules is described.

In other embodiments of the present disclosure, which can be used in combination with each or any of the above-mentioned aspects, the LDHC silencing molecule comprises short-interfering RNAs (siRNA) with a complementary or an antisense sequence to some or all isoforms of human LDHC-specific mRNA.

In some embodiments of the present disclosure, which can be used in combination with each or any of the above-mentioned aspects, the LDHC silencing molecule comprises vector-based short-hairpin RNAs (shRNA) with a specific complementary sequence to some or all isoforms of human LDHC-specific mRNA.

In other embodiments of the present disclosure, which can be used in combination with each or any of the above-mentioned aspects, the LDHC silencing molecule is administered in an amount effective to partially or fully degrade the LDHC mRNA. The effect of degrading the LDHC mRNA will reduce or eliminate the production of LDHC protein or LDHC antigen in the LDHC-expressing tumor or cancer cells.

In some embodiments of the present disclosure, which can be used in combination with each or any of the above-mentioned aspects, the siRNA is chemically modified to allow delivery of the naked siRNA into human cells. The chemical modifications can comprise, but are not limited to, incorporation of locked nucleic acids (LNA), phosphorothioate linkages, 2′-o-methyl, 2′-amine, 2′fluoro groups, and/or conjugation with cholesterol, folate, aptamer, and/or other oligonucleotide conjugates known to one of ordinary skill in the art.

In some embodiments of the present disclosure, which may be used in combination with each or any of the above-mentioned aspects, the method can also include administering LDHC-therapeutic interventions that can comprise inhibitors, antibodies, and/or nanobodies. For example, Oxamate and oxamate analogues can inhibit Lactate dehydrogenase enzymes (LDHA, LDHB, LDHC) with varying degrees of selectivity. N-Ethyl oxamate is a potent inhibitor with a high affinity for LDHC, while N-propyl oxamate is a selective inhibitor with a high degree of selectivity towards LDHC.

In other embodiments of the present disclosure, which can be used in combination with each or any of the above-mentioned aspects, the inhibitor, antibody and/or nanobody is administered in an amount effective to block the active site of LDHC but not the active site of the LDHA or LDHB. For example, the nanobody has a high affinity for the active site of LDHC and a low affinity for the active sites of LDHA and/or LDHB.

In some embodiments of the present disclosure, which can be used in combination with each or any of the above-mentioned aspects, the inhibitor, antibody and/or nanobody is administered in an amount effective to block or to interfere with the binding of LDHC to its substrate. For example, the nanobody has a high affinity for the substrate-binding site of LDHC and a low affinity for the substrate-binding sites of LDHA and/or LDHB.

In other embodiments of the present disclosure, which can be used in combination with each or any of the above-mentioned aspects, the LDHC targeting molecule is administered or delivered to the subject via the use of a delivery system.

In some embodiments of the present disclosure, which can be used in combination with each or any of the above-mentioned aspects, the LDHC targeting molecule is administered or delivered to the subject via the use of more than one type of delivery system.

In other embodiments of the present disclosure, which can be used in combination with each or any of the above-mentioned aspects, the delivery system can comprise a DNA-scaffold based delivery system.

In some embodiments of the present disclosure, which can be used in combination with each or any of the above-mentioned aspects, the DNA-scaffold based delivery system can include modifications.

In other embodiments of the present disclosure, which can be used in combination with each or any of the above-mentioned aspects, the delivery system can comprise lipid-based liposomes or lipoplexes. The lipid-based liposome and/or lipoplex delivery systems can be complexed with phospholipids including, but not limited to, cationic lipids such as 1,2-dioleoyloxy-3-trimethylammonium propane (DOTAP) and/or 1,2-di-o-octadecenyl-2-trimethylammonium propane (DOTMA) and/or neutral lipids such as 1,2-dioleoyl-sn-glycero-3-phosphoethanolamine (DOPE), zwitterionic 1,2-dioleoyl-sn-glycero-3-phosphatidylcholine (DOPC) or their modified forms.

In some embodiments of the present disclosure, which can be used in combination with each or any of the above-mentioned aspects, the delivery system can include polymer-based nanoparticles or nanoplexes.

In other embodiments of the present disclosure, which can be used in combination with each or any of the above-mentioned aspects, the polymer-based nanoparticles or nanoplexes can be complexed with polymers, including but not limited to, polyethyleneimine (PEI), poly-1-lysine (PLL), chitosan, inulin, protamine, gelatin, atelocollagen, cationic polypeptides, cyclodextran polymers, dendrimers, poly-lactide-co-glycolide (PLGA), polydimethylaminoethylmethacrylate (PDMAEMA) and/or their modified forms.

In some embodiments of the present disclosure, which can be used in combination with each or any of the above-mentioned aspects, the delivery system can comprise nanostructures.

In other embodiments of the present disclosure, which can be used in combination with each or any of the above-mentioned aspects, the delivery system comprising nanostructures can be complexed with targeting molecule(s). The complex can comprise, but is not limited to, neutral lipid-based nanoliposomes, stable nucleic acid lipid particles (SNALP), solid lipid nanoparticles (SLN), and/or dendrimers.

In some embodiments of the present disclosure, which can be used in combination with each or any of the above-mentioned aspects, the delivery system can include a biologically responsive delivery system.

In other embodiments of the present disclosure, which can be used in combination with each or any of the above-mentioned aspects, the biologically responsive delivery system can comprise biosignal-responsive crosslinkers. The biosignal-responsive crosslinkers can be capable of releasing encapsulated covalently conjugated siRNA/inhibitors and/or can activate cell penetrating peptides in response to tumor-specific stimuli such as low pH, hypoxia, changes in ATP concentration, and/or presence of enzymes specific to tumor microenvironments, for example, Matrix Metalloproteinases (MMPs).

In some embodiments of the present disclosure, which can be used in combination with each or any of the above-mentioned aspects, the delivery system can be modified with tumor homing peptides. The tumor homing peptides can allow selective binding to receptor molecules overexpressed on tumor cells. The tumor homing peptides can include, but are not limited to, arginine-glycine-aspartic acid (RGD) peptide and folate, or tumor homing antibodies or their fragments (antigen-binding Fab portion) to recognize tumor-specific antigens molecules on the surface of cancer cells.

In other embodiments of the present disclosure, which can be used in combination with each or any of the above-mentioned aspects, the delivery system can comprise cell- or tumor-derived extracellular vesicles/exosomes or their engineered forms.

In some embodiments of the present disclosure, which can be used in combination with each or any of the above-mentioned aspects, the composition is administered locally via intra-tumoral injection into solid tumors. This embodiment is especially useful for delivery systems that are not tumor-specific. The local administration can also be accomplished by any routes or methods known to one of ordinary skill in the art.

In other embodiments of the present disclosure, which can be used in combination with each or any of the above-mentioned aspects, the LDHC targeting molecule alone or the LDHC targeting molecule via a delivery system is administered systemically. The systemic administration can be accomplished by intraperitoneal or intravenous injection. In addition, systemic administration can also be accomplished by any routes or methods known to one of ordinary skill in the art.

In other embodiments of the present disclosure, which can be used in combination with each or any of the above-mentioned aspects, the method can also include administering non-specific, DNA damaging drugs and/or DNA damage repair inhibitors, in addition to LDHC targeting molecules. The non-specific, DNA damaging drugs and/or DNA damage repair inhibitors can include, but are not limited to, platinum-based alkylating agents (e.g. cisplatin, carboplatin etc) and Poly ADP-Ribose Polymerase (PARP) inhibitors (e.g. Olaparib, niraparib etc.).

The compositions described herein can be administered only one time to the subject or more than one time to the subject. Furthermore, when the compositions are administered to the subject more than once, a variety of regimens may be used, such as, but not limited to, one per day, once per week, once per month or once per year. The therapeutically effective amount and appropriate dosing regimens can be identified by routine testing in order to obtain optimal efficacy, while minimizing potential side effects. In addition, co-administration or sequential administration of other agents can be desirable.

In some embodiments, the compositions of the present disclosure can further comprise agents which improve the solubility, absorption, half-life, etc. Furthermore, the compositions of the present disclosure can further comprise agents that attenuate undesirable side effects and/or decrease any toxicity. Examples of such agents are described in a variety of texts, such as, but not limited to, Remington: The Science and Practice of Pharmacy (20th Ed., Lippincott, Williams & Wilkins, Daniel Limmer, editor).

In other embodiments of the present disclosure, the pharmaceutical compositions can further comprise a pharmaceutically acceptable carrier. Typically, the pharmaceutically acceptable carrier has no detrimental side effects or toxicity under the conditions of use. The nature of the pharmaceutically acceptable carrier may differ depending on the particular dosage form employed and other characteristics of the composition.

Examples of pharmaceutically acceptable carriers may include, but are not limited to, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil, which are is generally used in the art to which the present disclosure pertains. In addition, the compositions disclosed herein comprise diluents or excipients, such as fillers, extenders, binders, wetting agents, disintegrants or surfactants, and other pharmaceutically acceptable additives.

The pharmaceutical compositions of the present disclosure can be modified to prevent adverse reactions in the subject.

The pharmaceutical preparations of the present disclosure can be stored in any pharmaceutically acceptable form, including an aqueous solution, a frozen aqueous solution, a lyophilized powder, or any of the other forms described herein.

### EXAMPLES

### Example 1

### LDHC Expression in Breast Cancer

To confirm the testis-specific expression of LDHC, we interrogated publicly available databases and performed in-house qPCR for a wide range of normal tissue types. We verified that LDHC mRNA and protein expression is restricted to testis tissues with lack of or very low expression in other normal tissues (Supplementary FIG. 1). Analysis of the TOGA breast cancer dataset revealed a trend of increased LDHC expression in breast tumor tissue compared to normal tissue (FIG. 1A). The variability in expression levels prompted us to investigate LDHC expression across intrinsic molecular breast tumor subtypes. LDHC expression was significantly increased in basal-like tumors, the least favorable molecular subtype, compared to luminal A tumors, the most favorable subtype (FIG. 1B). Furthermore, high expression of LDHC, in particular in basal-like breast tumors, was associated with adverse overall (HR=1.33, p value=0.08) and disease-specific survival (HR=1.86, p value=0.002) (FIG. 10). These findings are in accordance with a pro-tumorigenic role for LDHC in less favorable subtypes such as basal-like tumors.

### Example 2

### LDHC Silencing Induces Giant Cell Formation

In order to investigate the role of LDHC in breast cancer, its expression was stably silenced in three basal-like breast cancer cell lines (MDA-MB-468, BT-549, MDA-MB-231) alongside one non-basal, luminal A breast cancer cell line (HCC-1500). LDHC mRNA and protein expression was significantly reduced in all cell lines using LDHC-specific shRNA, albeit at different efficiencies, with the highest knockdown of LDHC in MDA-MB-468 and HCC-1500 and to a lesser extent in MDA-MB-231 and BT-549 (FIG. 1D). Of note, no significant changes in LDHA and LDHB were observed upon LDHC silencing (Supplementary FIG. 2A). Silencing of LDHC significantly increased the number of giant cells and induced changes in the actin cytoskeleton whereby the ring-like distribution of filamentous actin (F-actin) stress fibers in shCTRL cells was replaced by a network of elongated actin filaments in a proportion of shLDHC cells (FIG. 1E).

### Example 3

### LDHC Silencing Induces Genomic Instability and Mitotic Catastrophe

Next, we investigated the presence and extent of genomic instability and nuclear aberrations as common features of giant cancer cells. We found that LDHC silencing increased the frequency and extent of polyploidy (4N) in MDA-MB-468 and BT-549 cells (FIG. 2A-2B) but not in breast cancer cells with inherent low levels of polyploidy (MDA-MB-231 and HCC-1500). MDA-MB-468 shLDHC cells displayed an increase in the proportion of cells with 8N, 10N and 12N polyploidy and decrease in 6N polyploidy (FIG. 2B). In addition, shLDHC cells displayed an increase in the presence of nuclear aberrations, including multinucleation (MNC), micronuclei (MN), nucleoplasmic bridges (NPB) and nuclear budding (NBUD) (FIG. 2C). As a countermeasure for increased genomic instability, cells can undergo mitotic catastrophe (MC), driving cells into an antiproliferative fate. In line with this protective mechanism, a significant proportion of shLDHC cells exhibit mitotic catastrophe, featuring multiple spindle assemblies and defects in spindle formation leading to lagging chromosomes and improper mitotic segregation (FIG. 2C). We were not able to validate these observations in the HCC-1500 cell line model due to the relatively small cell size impeding accurate microscopic assessment.

### Example 4

### LDHC Silencing Triggers Excessive DNA Damage and Microtubule Destabilization

In line with an increase in mitotic catastrophe, LDHC silencing markedly increased the expression of phospho-gamma-H2AX (y-H2AX) in all four cell line models, indicating the presence of excess DNA damage (FIG. 3A). Assessment of DNA damage sensors upstream of the DNA damage response (DDR) pathway demonstrated that expression of ATM, ATR, DNA-PKC and phospho-BRCA1 were downregulated by LDHC silencing, albeit to slightly different extents based on the cell line (Supplementary FIG. 2B). Furthermore, LDHC silencing disrupted mitotic spindle organization, a common feature of cells undergoing mitotic catastrophe. More specifically, we found an increase in α-tubulin degradation (FIG. 3B), a decrease in its acetylation of lysine residue 40 (FIG. 3C), and a more punctate staining in shLDHC cells (FIG. 3E), collectively suggesting an increase in microtubule destabilization. In addition, the expression of microtubule-associated protein 1B (MAP1B), involved in maintaining microtubule stability, was significantly downregulated by LDHC silencing in three out of four cell lines (FIG. 3D-3E). The lack of a decrease in MAP1B in MDA-MB-231 shLDHC cells suggests that alternative MAPs may be involved in driving microtubule destabilization in these cells. Together, these findings indicate that LDHC silencing promotes genomic instability and mitotic catastrophe concomitant with excessive DNA damage and mitotic spindle destabilization.

### Example 5

### Mitotic Catastrophe-Associated Cell Fates

The activation of mitotic catastrophe can drive cells towards either of two cell fates; mitotic cell death or mitotic slippage. Hence, we assessed whether LDHC silencing facilitates either cell fate, and determined the expression of key players of mitotic entry and exit.

### Increased Cell Death

LDHC silencing induced apoptosis in all four breast cancer cell lines, as demonstrated by an increase in caspase 3 cleavage (FIG. 4A, Supplementary FIG. 2C). Furthermore, flow cytometric analysis revealed an increase in the proportion of early (AnnexinV positive, PI negative) and/or late (AnnexinV positive, PI positive) apoptotic cells after LDHC silencing (FIG. 4A, Supplementary FIG. 2D-2E).

### Aberrant Mitosis and Loss of Survival

Next, we investigated whether LDHC silencing is associated with mitotic slippage whereby cells prematurely exit mitosis by analyzing the cell cycle distribution of synchronized cells for up to 12 hours (FIG. 4B, Supplementary FIG. 3A-B). In comparison to MDA-MB-468 shCTRL cells, shLDHC cells demonstrated rapid transition from G0/G1 to S and G2/M phase (2 h and 8 h respectively), and mitotic slippage with premature mitotic exit from the G2/M phase into the next cell cycle (12 h) (FIG. 4B). In addition, we found an increase in the proportion of shLDHC cells likely undergoing mitotic cell death (34% vs 21% sub-G1 shCTRL at 12 h) (Supplementary FIG. 3C). Similarly, analysis of BT-549 shLDHC cells revealed rapid transition from G0/G1 to S phase (Supplementary FIG. 3B) and increased cell death (12 h) (Supplementary FIG. 3C). Of note, mitotic slippage in BT-549 shLDHC cells was preceded by G0/G1 arrest in a likely attempt to repair DNA damage, and the cells displayed a significantly higher number of polyploid cells (12 h), suggesting sustained defective mitosis(Supplementary FIG. 3B). Both cell line models support the hypothesis that LDHC silencing induces mitotic dysregulation and subsequent apoptosis. Moreover, analysis of asynchronous populations of MDA-MB-468 and BT-549 cells (FIG. 4C) provided further evidence for shLDHC-associated mitotic slippage with an increase in actively replicating polyploid cells (polyploid Edu+). Additional analyses of all four cell line models (FIG. 4C, Supplementary FIG. 4A) revealed a shLDHC-associated shift in the proportion of cells in G0/G1 (2N, EdU-negative) and G2/M (4N, EdU-negative) with MDA-MB-468 and HCC-1500 shLDHC cells also showing an increase in sub-G1 apoptotic cells. Together, these results suggest that silencing LDHC is associated with both mitotic slippage and arrest, followed by cell death.

To gain insight into the molecular mechanisms driving progression through mitosis, we assessed the activation status of the cyclin B1-cdc2 complex in the MDA-MB-468 cell line that demonstrated high LDHC silencing efficiency and a robust phenotype (FIG. 4D). Analysis of the expression of cyclin B1 and of inactive phosphorylated cdc2 (Tyr15 phospho-cdc2) revealed remarkable differences in expression dynamics in shLDHC cells compared to shCTRL cells over a time period of 48 hours (approximately two cycles of mitotic entry). Both control and LDHC-silenced cells displayed the characteristic pre-mitotic peak in cyclin B1 expression at 12 h post synchronization, however, shLDHC cells showed an earlier decline in phospho-cdc2 (6-16 vs 12-16 h in shCTRL) in accordance with a more rapid G2/M transition and initiation of mitosis. Subsequently, shLDHC cells displayed an earlier second peak in cyclin B1 expression followed by cdc2 dephosphorylation (28 h vs 40 h in shCTRL), indicating that shLDHC cells prematurely entered the second round of mitosis. Of note, the earlier peak of cyclin B1 and phospho-cdc2 expression in shLDHC cells are followed by a slower rate of degradation. Moreover, their expression does not decline to baseline levels after the first round of mitosis (20-24 h). In concordance with our cell cycle profiling data, these observations suggest that LDHC silencing may result in two cell subpopulations, whereby one subset of cells undergoes mitotic slippage and the other experiences prolonged pre-mitotic arrest with likely subsequent cell death as a result of excessive, unrepaired DNA damage. Indeed, it is well known that the balance of two opposing networks, involving cyclin B1 degradation and caspase cleavage, determine mitotic cell fate. Analysis of phospho-γ-H2AX levels demonstrated a high level of DNA damage in shLDHC cells at 0 h and at the mitotic phases (24 h and 48 h) (FIG. 4D). Furthermore, assessment of caspase 3 cleavage (FIG. 4D) provides evidence of a sustained increase in shLDHC cell death throughout the cell cycle (6-12 h and 20 h onwards) and in subsequent cell cycles (48 h).

In addition to undergoing apoptosis, mitotic catastrophe can also drive cells to enter a state of senescence as observed in HCC-1500 shLDHC cells but not in the other three cell lines (Supplementary FIG. 4B). Strikingly, silencing of LDHC significantly decreased the colony-formation ability, hence long-term survival, of all four breast cancer cell lines (FIG. 4E, Supplementary FIG. 4C).

### Example 6

### LDHC Silencing Dysregulates Multiple Cell Cycle Checkpoints

Based on our observations of increased mitotic dysregulation by LDHC silencing, we explored potential defects in G1/S, intra-S, G2/M and Spindle Assembly checkpoint (SAC) regulators using the MDA-MB-468 cell line model. Differential gene expression and gene ontology analysis of 84 cell cycle-related genes revealed an enrichment of genes involved in dysregulated DNA damage response and impaired mitotic fidelity in LDHC-silenced cells (FIG. 5A, Supplementary Table 2).

Furthermore, we found that LDHC silencing altered the protein expression of cell cycle regulators at multiple checkpoints. For instance, the expression of the G1/S checkpoint regulators Cyclin D1, Cyclin E2 and Cyclin-dependent kinase (CDK)-6 was downregulated in shLDHC cells (FIG. 5B), suggesting that less cells reside in the G1 phase or that the G1 phase is shortened, which is in line with our cell cycle profile observations (FIG. 4C). The observed upregulation of INK4 type CDK inhibitors (p16, p18) and associated downregulation of p53 and its downstream targets p27 and p21 further supports the likelihood of G1/S checkpoint dysregulation in shLDHC cells. In addition, LDHC silencing increased the expression of cyclin-dependent kinase subunit 2 (CKS2), likely mediating an override of the intra-S phase checkpoint in the presence of replication stress and facilitating transition from G2 to M phase (FIG. 5B, Supplementary Table 2). Expression analysis of the negative G2/M checkpoint regulators Wee1 and Myt1 demonstrated a decrease in total and active phosphorylated Wee1 (Ser642), and conversely an increase in total and inactive phosphorylated Myt1 (Ser83) in shLDHC cells, supporting our previous observations that LDHC silencing promotes mitotic entry. On the other hand, we found an increase in phosphorylated checkpoint proteins Chk1 (Ser345) and Chk2 (Thr68), indicating the presence of single- and double-strand DNA breaks, that in turn inactivate Cdc25C by phosphorylation at Ser216, resulting in G2/M arrest. Collectively, these findings corroborate the presence of two shLDHC cell subpopulations; one population that undergoes G2/M arrest and another that undergoes checkpoint adaptation and slippage.

Further evidence for the existence of two shLDHC cell populations was provided by the assessment of mitotic/SAC regulators such as Aurora A and Mad2L1. We found that total and phosphorylated Aurora A expression were significantly reduced in shLDHC cells (FIG. 5B, Supplementary Table 2), and as such may result in the loss of an active SAC with premature transition from metaphase into anaphase. In addition, expression of Mad2L1 was upregulated after LDHC silencing, possibly triggering cells to undergo long-term SAC activation followed by mitotic slippage (FIG. 5B, Supplementary Table 2). In contrast, we observed a slight decrease in the expression of the kinetochore-associated protein BubR1 in shLDHC cells, thus impairing accurate spindle attachment and chromosome segregation. Although the cellular localization of BubR1 did not differ in the pro-metaphase between shCTRL and shLDHC cells, its expression at the kinetochores and at segregating chromosomes in the metaphase and early anaphase was dysregulated in shLDHC cells, indicating defective sister chromatid segregation and prevention of anaphase onset (FIG. 5C). In line with this, the majority of shLDHC cells in anaphase remained BubR1 positive whereas shCTRL cells completely lost BubR1 expression going from metaphase to anaphase.

In conclusion, assessment of the expression of cell cycle regulators in the MDA-MB-468 cell line confirmed the presence of two cell subpopulations after LDHC silencing: one subpopulation undergoing mitotic arrest at the G2/M and/or SAC checkpoint, and another undergoing mitotic slippage. This phenotype of mitotic dysregulation upon LDHC silencing was observed in all four breast cancer cell lines, however, the molecular mechanistic underpinnings of these observations may vary between cell lines based on their genetic landscape.

### Example 7

### LDHC-Silencing Sensitizes Cancer Cells to DNA Damage Repair Inhibitors and DNA Damage Inducers

DNA damage repair inhibitors and DNA damage inducing agents, including the Poly ADP-ribose polymerase (PARP)-inhibitor olaparib and cisplatin, are widely used anti-cancer drugs with particular benefit to patients with defects in DDR pathways such as BRCA1/2-positive or ‘BRCAness’ basal breast cancer patients. Since we demonstrated that LDHC silencing increases DNA damage accumulation in breast cancer cells with alterations in the expression of DNA damage sensors upstream of the DDR pathway, we explored the sensitivity of shLDHC cells to olaparib and cisplatin. Treatment with either drug further induced apoptosis in giant shLDHC cells compared to shCTRL cells (FIG. 6A, Supplementary FIG. 5A-C). In accordance, olaparib and cisplatin treatment augmented the expression of cleaved caspase 3 in shLDHC cells (FIG. 6B). Moreover, we observed a significant increase in DNA damage after olaparib and cisplatin treatment, albeit at similar levels in both shCTRL and shLDHC treated cells (FIG. 6C). However, shLDHC cells with excessive DNA damage displayed apoptotic nuclear features such as highly condensed DNA and nuclear fragmentation. Finally, the colony formation ability of shLDHC cells was further compromised in a cell-line dependent manner, by about 2-fold after treatment with olaparib and greater than 4-fold after treatment with cisplatin, compared to shCTRL treated cells (FIG. 6D, Supplementary FIG. 5D). The MDA-MB-468 and BT-549 shLDHC cells were the most sensitive and the MDA-MB-231 shLDHC cells were the least sensitive to treatment.

SUPPLEMENTARY TABLE 1
List of Antibodies

| Antibody | Catalog No. | Manufacturer | Host | Concentration |
|---|---|---|---|---|
| Acetylated alpha- | 5335 | Cell Signaling | Rabbit | 1:200 (IF) |
| tubulin (Lys40) | | | | |
| Alpha-tubulin | ab52866 | Abcam | Rabbit | 1:1000 (WB), |
| | | | | 1:200 (IF) |
| ATM (D2E2) | 2873 | Cell Signaling | Rabbit | 1:1000 (WB) |
| ATR (E1S3S) | 13934 | Cell Signaling | Rabbit | 1:1000 (WB) |
| Aurora A | 14475 | Cell Signaling | Rabbit | 1:1000 (WB) |
| Beta-Actin | 4970 | Cell Signaling | Rabbit | 1:1000 (WB) |
| Beta-tubulin | 2128 | Cell Signaling | Rabbit | 1:1000 (WB), |
| | | | | 1:200 (IF) |
| BubR1 | ab28193 | Abcam | Sheep | 1:500 (WB), |
| | | | | 1:100 (IF) |
| Caspase 3 | 9662 | Cell Signaling | Rabbit | 1:1000 (WB) |
| Cdc25C | 4688 | Cell Signaling | Rabbit | 1:1000 (WB) |
| CDK6 | 3136 | Cell Signaling | Mouse | 1:1000 (WB) |
| CKS2 | ab155078 | Abcam | Rabbit | 1:1000 (WB) |
| Cyclin B1 | 12231 | Cell Signaling | Rabbit | 1:1000 (WB) |
| Cyclin D1 | 2978 | Cell Signaling | Rabbit | 1:1000 (WB) |
| Cyclin E2 | 4132 | Cell Signaling | Rabbit | 1:1000 (WB) |
| DNA-PKcs | 38168 | Cell Signaling | Rabbit | 1:1000 (WB) |
| (E6U3A) | | | | |
| LDHC | ab52747 | Abcam | Rabbit | 1:500 (WB) |
| Mad2L1 | ab97777 | Abcam | Rabbit | 1:1000 (WB) |
| MAP1B | ab11266 | Abcam | Mouse | 1:200 (IF) |
| Myt1 | 4282 | Cell Signaling | Rabbit | 1:1000 (WB) |
| P16 | 80772 | Cell Signaling | Rabbit | 1:1000 (WB) |
| P18 | 2896 | Cell Signaling | Mouse | 1:1000 (WB) |
| P21 | 2947 | Cell Signaling | Rabbit | 1:1000 (WB) |
| P27 | 3686 | Cell Signaling | Rabbit | 1:1000 (WB) |
| P53 | 2527 | Cell Signaling | Rabbit | 1:1000 (WB) |
| Phalloidin-Alexa | A12380 | Thermo Fisher | | 1:100 (IF) |
| Fluor 568 | | | | |
| Phospho-BRCA1 | 9009 | Cell Signaling | Rabbit | 1:1000 (WB) |
| (Ser1524) | | | | |
| Phospho-gamma | ab11174 | Abcam | Rabbit | 1:1000 (WB), |
| H2AX (S139) | | | | 1:200 (IF) |
| Phospho-cdc2 | 4539 | Cell Signaling | Rabbit | 1:1000 (WB) |
| (Tyr15) | | | | |
| Phospho-Cdc25C | 4901 | Cell Signaling | Rabbit | 1:1000 (WB) |
| (Ser216) | | | | |
| Phospho-Chk1 | 2348 | Cell Signaling | Rabbit | 1:1000 (WB) |
| (Ser345) | | | | |
| Phospho-Chk2 | 2197 | Cell Signaling | Rabbit | 1:1000 (WB) |
| (Thr68) | | | | |
| Phospho-Myt1 | 4281 | Cell Signaling | Rabbit | 1:1000 (WB) |
| (Ser83) | | | | |
| Phospho-Wee1 | 4910 | Cell Signaling | Rabbit | 1:1000 (WB) |
| (Ser642) | | | | |
| Wee1 | 13084 | Cell Signaling | Rabbit | 1:1000 (WB) |

WB = western blotting;
IF = immunofluorescence

SUPPLEMENTARY TABLE 2
Differentially expressed cell cycle genes. Differentially
expressed genes after LDHC silencing in MDA-MB-468 cells as
determined by the Cell Cycle RT2 Profiler qPCR array.

| Gene Symbol | Fold Regulation |
|---|---|
| AURKA | 4.60 |
| AURKB | 2.80 |
| BIRC5 | 4.28 |
| BRCA1 | 2.24 |
| BRCA2 | 2.83 |
| CCNA2 | 4.03 |
| CCNB1 | 3.27 |
| CCNB2 | 2.42 |
| CCND3 | 2.77 |
| CCNF | 4.15 |
| CDC20 | 3.29 |
| CDC25A | 2.90 |
| CDC25C | 3.50 |
| CDC6 | 2.32 |
| CDK1 | 3.40 |
| CDK2 | 2.29 |
| CDKN3 | 2.21 |
| CHEK2 | 2.39 |
| CKS2 | 2.05 |
| E2F1 | 2.51 |
| GTSE1 | 3.58 |
| KPNA2 | 2.36 |
| MAD2L1 | 2.50 |
| MKI67 | 4.12 |
| RAD51 | 2.24 |
| STMN1 | 2.06 |

SUPPLEMENTARY TABLE 3
Summary of observed characteristics in LDHC-silenced
breast cell lines

| | MDA-MB- | | MDA- | |
|---|---|---|---|---|
| | 468 | BT-549 | MB-231 | HCC-1500 |
| | Parental cell properties | | | |
| Molecular subtype | Basal-like | Basal-like | Basal-like | Luminal A |
| BRCA | Wild-type | Wild-type | Wild-type | Wild-type |
| p53 | mutant | mutant | mutant | negative |
| Rb | negative | negative | positive | positive |
| | LDHC silencing phenotypes | | | |
| Giant cells | Increase | Increase | Increase | Increase |
| Polyploidy | Increase | Increase | low baseline | low baseline |
| | | | level, | level, |
| | | | no change | no change |
| DNA damage | Increase | Increase | Increase | Increase |
| Apoptosis | Increase | Increase | Increase | Increase |
| Long-term survive/ | Decrease | Decrease | Decrease | Decrease |
| Cinnogenicity | | | | |
| Microtubule instability | Increase | Increase | Increase | Increase |
| Cell population in G1 | Decrease | Decrease | Decrease | Decrease |
| Cell population | Increase | Increase | Increase | Increase |
| in G2/M | | | | |
| Mitotic slippage | Increase | Increase | ND | ND |
| Cell cycle arrest | Increase | Increase | ND | ND |
| Senescence | No change | No change | No change | Increase |
| Sensitivity to Cisplatin | Increase | Increase | Increase | No change |
| Sensitivity to Olaparib | Increase | Increase | Increase | Increase |

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of treating an LDHC-expressing cancer in a subject in need thereof, the method comprising administering to the subject an effective amount of a composition comprising an LDHC targeting molecule.

2. The method of claim 1, wherein the LDHC targeting molecule is administered or delivered to the subject via the use of a delivery system.

3. The method of claim 1, wherein the LDHC silencing molecule comprises short-interfering RNAs (siRNA).

4. The method of claim 3, wherein the siRNA is chemically modified to allow delivery of the naked siRNA into the subject's cells.

5. The method of claim 1, wherein the LDHC silencing molecule comprises vector-based short-hairpin RNAs (shRNA).

6. The method of claim 1, wherein the composition comprises an inhibitor, a nanobody, and/or antibody.

7. The method of claim 6, wherein the composition comprises at least one inhibitor.

8. The method of claim 6, wherein the composition comprises at least one nanobody.

9. The method of claim 6, wherein the inhibitor, antibody and/or nanobody is specific for the active site of LDHC or the substrate-binding site of LDHC.

10. The method of claim 1, wherein the delivery system comprises a DNA-scaffold based delivery system.

11. The method of claim 1, wherein the delivery system comprises lipid-based liposomes or lipoplexes.

12. The method of claim 1, wherein the delivery system comprises polymer-based nanoparticles or nanoplexes.

13. The method of claim 12, wherein the polymer-based nanoparticles or nanoplexes are complexed with polymers.

14. The method of claim 1, wherein the delivery system comprises nanostructures.

15. The method of claim 1, wherein the delivery system includes a biologically responsive delivery system.

16. The method of claim 15, wherein the biologically responsive delivery system comprises biosignal-responsive crosslinkers.

17. The method of claim 1, wherein the delivery system is modified with tumor homing peptides.

18. The method of claim 1, wherein the delivery system comprises cell- or tumor-derived extracellular vesicles/exosomes or their engineered forms.

19. The method of claim 1, wherein the LDHC silencing molecule is administered to a subject with LDHC-expressing cancer, in an amount effective to partially or fully degrade the LDHC mRNA.

20. The method of claim 1, wherein the LDHC silencing molecule is administered to a subject with LDHC-expressing cancer, in an amount effective to reduce or eliminate the production of LDHC protein or LDHC antigen in tumors or cancer cells.

21. A method of preventing the progression, decreasing incidence of, and/or decreasing severity of an LDHC-expressing tumor in a subject in need thereof, the method comprising administering to the subject a composition comprising an effective amount of LDHC targeting molecules.

22. The method of claim 21, wherein the subject is human.

23. The method of claim 21, wherein the composition is administered systemically and/or locally.

24. The method of claim 21, wherein the composition comprises a DNA damaging drug and/or DNA damage repair inhibitor.
